(19) United States
(12) Patent Application Publication
(10) Pub. No.: US 2012/0231499 A1
(43) Pub. Date: Sep. 13, 2012

(54) HIGH-MOLECULAR-WEIGHT RECOMBINANT SILK OR SILK-LIKE PROTEIN AND MICRO- OR NANO-SIZED SPIDER SILK OR SILK-LIKE FIBER PRODUCED THEREFROM

(76) Inventors: Sang Yup Lee, Daejeon (KR); Xiaoxia Xia, Daejeon (KR); Zhi Gang Qian, Daejeon (KR); Jeong Wook Lee, Daejeon (KR); Young Hwan Park, Seoul (KR)

(21) Appl. No.: 13/124,818

(22) PCT Filed: Mar. 11, 2011
(86) PCT No.: PCT/KR11/01730

§ 371 Date: Aug. 11, 2011

(51) Int. Cl. C12P 21/02; D01D 5/06; C07K 14/00

(52) U.S. Cl. 435/69.1; 530/353; 264/178 F

(57) ABSTRACT

A high-molecular-weight recombinant silk or silk-like protein having a molecular weight which is substantially similar to that of native silk protein, and a micro- or nano-sized spider silk or silk-like fiber having improved physical properties, produced therefrom. The recombinant silk or silk-like protein according to the invention has high molecular weight, like dragline silk proteins from spiders, while a fiber produced therefrom has excellent physical properties compared to a fiber produced from native silk protein. Thus, the recombinant silk or silk-like protein and the spider silk or silk-like fiber produced therefrom will be highly useful in various industrial applications, including bioengineering applications and medical applications.

### CROSS-REFERENCE TO RELATED APPLICATIONS

The present application claims priority under 35 U.S.C. 119 to Korean patent application No. 10-2010-0021934, filed on Apr. 11, 2010.

### BACKGROUND OF THE INVENTION

1. Technical Field

The present invention relates to a high-molecular-weight recombinant silk or silk-like protein having a molecular weight which is substantially similar to that of native silk protein, and to a micro- or nano-sized spider silk or silk-like fiber having improved physical properties, produced therefrom.

2. Description of the Related Art

Spider dragline silk that is used by spiders as the safety line and the web frame is very strong and elastic. Indeed, spider dragline silk is five times stronger by weight than steel, and three times tougher than the top quality man-made fiber Kevlar (Gosline, J. M. et al., J. Exp. Biol., 202:3295, 1999; Vollrath, F. & Knight, D. P., Nature 410: 541, 2001). Accordingly, spider dragline silk has received a great deal of attention as a material which can be used in various industrial applications. Also, spider dragline silk is biocompatible and biodegradable, and thus is envisioned in many biomedical applications. Unfortunately, native dragline silk cannot be conveniently obtained by farming spiders, because it is highly territorial and aggressive. Thus, there have been many efforts to produce recombinant dragline silk proteins (Lazaris, A. et al., Science, 295:472, 2002; Teule, F. et al., Nat. Protoc., 4: 341, 2009; Arcidiacono, S. et al., Macromolecules, 35: 1262, 2002; Brooks, A. E. et al. Biomacromolecules, 9: 1506, 2008; Heim, M., Keerl, D. & Scheibel, T., Angew. Chem. Int. Ed. Engl., 48: 3584, 2009; Fahnestock, S. R. et al., Rev. Mol. Biotechnol., 74: 105, 2000; Scheller, J. et al., Nat. Biotechnol., 19: 573, 2001; Widmaier, D. M. et al. Mol. Syst. Biol., 5: 309, 2009).

All the spiders studied have evolved dragline silk proteins having a molecular weight of 250-320 kDa. However, the largest of the dragline silk proteins that have been synthesized in E. coli has a molecular weight of 163 kDa (Fahnestock S. R. & Irwin S. L., Appl. Microbiol. Biotechnol., 47:23, 1997), which corresponds to half the molecular weight of the dragline silk produced by spiders. The reason why the production of a larger dragline silk protein was not reported is believed to contribute to a problem of non-homogeneity caused by an error occurred during a protein synthesis process.

Accordingly, the present inventors have made many efforts to provide a recombinant dragline silk protein which has a high molecular weight, like a dragline silk protein produced from spiders, and has physical properties similar to or better than native silk protein when being made into a fiber. As a result, the present inventors have found that a spider silk fiber having physical properties better than a conventional native spider silk fiber can be produced by co-expressing glycine tRNA in bacteria such as E. coli to produce a recombinant silk protein having a high molecular weight of 284.9 kDa or more and then spinning the recombinant silk protein, thereby completing the present invention.

### DISCLOSURE OF INVENTION

It is an object of the present invention to provide a recombinant silk protein which has a high molecular weight, like native spider dragline silk proteins.

Another object of the present invention is to provide a spider silk fiber having improved physical properties, spun from a high-molecular-weight recombinant silk protein.

In order to accomplish the above objects, the present invention provides a high-molecular-weight recombinant silk or silk-like protein having a structure in which a peptide having a glycine content of 10% or more is repeated 64-160 times.

In addition, the present invention provides a high-molecular-weight recombinant silk protein having a structure in which a peptide of SEQ ID NO: 1 is repeated 64-160 times, the recombinant silk protein having a molecular weight of 192.8-482 kDa.

Also, the present invention provides a method for preparing a high-molecular-weight recombinant silk or silk-like protein, the method comprising co-expressing a gene encoding said recombinant silk or silk-like protein with a nucleotide sequence encoding glycine tRNA.

Further, the present invention provides a method for producing a micro-sized or nano-sized spider silk or spider silk-like fiber, the method comprising spinning a solution containing said high-molecular-weight recombinant silk or silk-like protein.

In addition, the present invention provides a micro-sized or nano-sized spider silk or spider silk-like fiber produced by said method.

In addition, the present invention provides a method for producing a micro-sized or nano-sized spider silk fiber, the method comprising spinning a solution containing a recombinant silk protein, wherein the recombinant silk protein having a structure in which a peptide of SEQ ID NO: 1 is repeated 64-160 times, the recombinant silk protein having a molecular weight of 192.8-482 kDa.

Moreover, the present invention provides a micro-sized or nano-sized spider silk fiber produced by said method.

### BRIEF DESCRIPTION OF THE DRAWINGS

FIG. 1 shows a system for expression of a recombinant silk protein and the amino acid sequence of the repeating unit of the recombinant silk protein.

FIG. 2 is a photograph showing the molecular weights of recombinant silk proteins of 16-mer, 32-mer, 64-mer and 96-mer, separated on 10% SDS-PAGE gel.

FIG. 3 is a graphic diagram showing stress-strain curves of fibers spun from 20% (w/v) recombinant silk protein solutions using a wet spinning method.

FIG. 4a-4c are graphic diagrams showing the breaking strain, tenacity and Young's modulus of fibers spun from 20% (w/v) recombinant silk protein solutions using a wet spinning method.

FIG. 5a-5c are graphic diagrams showing the breaking strain, tenacity and Young's modulus of fibers as function of the recombinant silk protein concentrations of dope solutions.

### BEST MODE FOR CARRYING OUT THE INVENTION

Unless defined otherwise, all technical and scientific terms used herein have the same meaning as commonly understood by one of ordinary skill in the art to which the invention pertains. Generally, the nomenclature used herein and the experiment methods are those well known and commonly employed in the art.

The definition of main terms used in the detailed description of the invention is as follows.

As used herein, the term “silk protein” refers to a synthetic silk protein that approximates the molecular and structural profile of native silk proteins and is biosynthesized by a recombinant protein production method. Examples of the silk protein include dragline silk, silk fibroin and flagelliform silk proteins. As used herein, the term “silk-like protein” refers to a protein which comprises as a repeating unit a peptide having a glycine content of 10% or more and is produced by, for example, a recombinant protein production method, like the silk protein. Examples of the silk-like protein include elastin, byssus, and collagen.

As used herein, the term “spider silk fiber” refers to a fiber which is produced from a synthetic recombinant silk protein and is substantially similar to a native spider silk fiber. The term “spider silk-like fiber” refers to a fiber which is produced from a synthetic recombinant silk-like protein and has physical properties similar to a spider silk protein.

As used herein, the term “recombinant protein” refers to a protein produced by expression of a nucleic acid sequence which is incorporated into a vector, i.e., e.g., an autonomously replicating plasmid or virus, or into the genomic DNA of a host cell, or which exists as a separate molecule in the host cell.

As used herein, the term “host cell” refers to any cell capable of expressing a functional gene and/or gene product introduced from another cell or organism.

In one aspect, the present invention is directed to a high-molecular-weight recombinant silk or silk-like protein having a structure in which a peptide having a glycine content of 10% or more is repeated 64-160 times.

In the present invention, the peptide having a glycine content of 10% or more, which constitutes the silk protein or silk-like protein, is preferably a repeating peptide constituting a protein selected from the group consisting of dragline silk protein, elastin, silk fibroin, byssus, flagelliform silk protein and collagen. Amino acid sequences of SEQ ID NOS: 1 to 4 are repeating peptides of dragline silk protein, amino acid sequences of SEQ ID NOS: 5-7 are repeating peptides of eastin, an amino acid sequence of SEQ ID NO: 8 is a repeating peptide of silk fibroin, an amino acid sequence of SEQ ID NO: 9 is a repeating peptide of byssus, and an amino acid sequence of SEQ ID NO: 10 is a repeating peptide of flagelliform silk protein, and amino acid sequences of SEQ ID NOS: 11 and 12 are repeating peptides of collagen.

SEQ ID NO: 1:
NH₂-SGRGGLGGQGAGMAAAAAMGGAGQGGYGGLGSQGT-COOH
SEQ ID NO: 2:
NH₂-GPGQQ-COOH
SEQ ID NO: 3:
NH₂-GPGGY-COOH
SEQ ID NO: 4:
NH₂-GGYGPGS-COOH
SEQ ID NO: 5:
NH₂-GVGVP-COOH
SEQ ID NO: 6:
NH₂-VPGG-COOH
SEQ ID NO: 7:
NH₂-APGVGV-COOH
SEQ ID NO: 8:
NH₂-GAGAGS-COOH
SEQ ID NO: 9:
NH₂-GPGGG-COOH
SEQ ID NO: 10:
NH₂-GPGGX-COOH
SEQ ID NO: 11:
NH₂-GAPGAPGSQGAPGLQ-COOH
SEQ ID NO: 12:
NH₂-GAPGTPGPQGLPGSP-COOH

In the present invention, it was demonstrated that a recombinant silk protein prepared to comprise 64 repeats of the amino acid sequence set forth in SEQ ID NO: 1 (hereinafter referred to as “64-mer”) has a molecular weight of about 192.8 kDa, thus making it possible to produce a recombinant silk protein having a higher molecular weight than the existing largest dragline silk protein (163 kDa) synthesized in E. coli. In addition, it was found that a recombinant silk protein prepared to comprise 96 repeats of the amino acid sequence set forth in SEQ ID NO: 1 (hereinafter referred to as “96-mer”) has a molecular weight reaching 284.9 kDa which is substantially similar to the molecular weight of native silk proteins (250-320 kDa) obtained from spiders.

However, recombinant silk proteins comprising 160 repeats or more of the peptide sequence cannot be synthesized by E. coli. Thus, the recombinant silk or silk-like protein according to the present invention preferably has a structure in which the peptide is preferably repeated 64-160 times, more preferably 80-160 times, and even more preferably 96-160 times.

In the present invention, the amino acid sequences that are used as repeating units are not limited to the exact sequences of SEQ ID NOS: 1 to 12. The amino acid sequences indicated herein also comprise variants. Thus, the amino acid sequences of the proteins of the present invention also encompass all sequences differing from the herein-disclosed sequences by amino acid insertions, deletions, and substitutions.

Preferably, amino acid “substitutions” are the result of replacing one amino acid with another amino acid having similar structural and/or chemical properties, i.e., conservative amino acid replacements. Amino acid substitutions may be made on the basis of similarity in polarity, charge, solubility, hydrophobicity, hydrophilicity, and/or the amphipathic nature of the residues involved. For example, nonpolar (hydrophobic) amino acids include alanine, leucine, isoleucine, valine, proline, phenylalanine, tryptophan, and methionine; polar neutral amino acids include glycine, serine, threonine, cysteine, tyrosine, asparagine, and glutamine; positively charged (basic) amino acids include arginine, lysine, and histidine; and negatively charged (acidic) amino acids include aspartic acid and glutamic acid.

“Insertions” or “deletions” in the repeating unit are typically in the range of about 1 to 5 amino acids, and preferably about 1, 2 or 3 amino acids. Amino acid additions in the repeating unit are typically less than 100, preferably less than 80, more preferably less than 50, most preferably less than 20 amino acids, which are inserted into the repeating unit of the present invention and added on and/or inserted into the protein of the present invention. It is noted that only those additions are contemplated in the present invention, which do not negatively affect the characteristics of the protein disclosed herein.

The variation allowed may be experimentally determined by systematically making insertions, deletions, or substitutions of amino acids in a protein using recombinant DNA techniques and assaying the resulting recombinant variants for activity. This does not inquire more than routine experiments for a person skilled in the art.

Accordingly, the protein of the present invention comprises as a repeating unit an amino acid sequence having a homology of at least 90% with SEQ ID NO: 1. As used herein, the phrase “having a homology of at least 90%” means that the protein has an identity of 91, 91.5, 92, 92.5, 93, 93.5, 94, 94.5, 95, 95.5, 96, 96.5, 97, 97.5, 98, 98.5, 99 or 99.5% with the sequence of SEQ ID NO: 1. The term “homology” refers to the degree of similarity between two amino acid sequences. Homologous proteins are those that are similar in sequence and function. Homology comparisons can be conducted by eye, or more usually, with the aid of readily available sequence comparison programs. These commercially available computer programs can calculate percent homology between two or more sequences (Wilbur, W. J. & Lipman, D. J., Proc. Natl. Acad. Sd. USA., 80:726, 1983).

In the present invention, the recombinant silk protein or the recombinant silk-like protein is preferably prepared by co-expressing glycine tRNA in bacteria. Accordingly, in another aspect, the present invention relates to a method for preparing the recombinant silk or silk-like protein, the method comprising co-expressing a gene encoding the recombinant silk protein or silk-like protein with a nucleotide sequence encoding glycine tRNA. Herein, the bacteria may be E. coli. Namely, the preparation of high-molecular-weight silk proteins has not been reported in the prior art; however, in the present invention, a recombinant silk protein having a high molecular weight of 192.8 kDa or more was prepared by co-expressing a gene encoding a silk protein, having as a repeating unit an amino acid sequence of SEQ ID NO: 1, with a nucleotide sequence encoding glycine tRNA in bacteria such as E. coli. Thus, the inventive silk protein having as a repeating unit the amino acid sequence of SEQ ID NO: 1 is characterized in that it has a molecular weight of 192.8-482 kDa.

In another aspect, the present invention is directed to a spider silk fiber or spider silk-like fiber having improved physical properties, produced by spinning the high-molecular-weight recombinant silk protein or silk-like protein.

In the present invention, a micro-sized or nano-sized spider silk fiber can be produced by spinning a dope solution containing the high-molecular-weight recombinant silk protein or silk-like protein through a spinneret.

As used herein, the term “dope solution” refers to any liquid mixture that contains silk protein and is amenable to extrusion for the formation of a spider silk fiber or film casting. Dope solutions may also contain, in addition to protein monomers, higher order aggregates including, for example, dimers, trimers, and tetramers. Normally, dope solutions are aqueous solutions of pH 4.0-12.0 and have less than 40% (w/v) organics or chaotropic agents. Preferably, the dope solution does not contain any organic solvents or chaotropic agents, but may include additives to enhance preservation, stability, or workability of the solution.

In the present invention, the dope solution preferably comprises 20-80% (w/v) of a recombinant silk protein.

In addition, the dope solution is preferably wet-spun in a liquid bath. Preferably, the liquid bath contains a liquid selected from the group consisting of methanol, ethanol, isopropanol, acetonitrile, water and aqueous ammonium sulfate.

Meanwhile, the diameter of the spider silk fiber can be determined by the diameter of the spinneret. The diameter of the spider silk fiber may be, for example, 0.650 μm, but the scope of the present invention is not limited thereto.

In one Example of the present invention, the physical properties (e.g., tenacity, Young's modulus and breaking strain) of the spider silk fiber produced by wet spinning were measured. The measurement results indicated that a spider silk fiber having improved physical properties can be provided using the recombinant silk protein according to the present invention. Particularly, a fiber produced from a recombinant silk protein of 96-mer showed a tenacity of 508±108 MPa and a breaking strain of 15±5%, which are comparable to the values reported for native N. clavipes dragline silk (740-1,200 MPa and 18-27%). In particular, the Young's modulus of the 96-mer fiber was 21±4 GPa corresponding to twice that of the native dragline silk (11-14 GPa). The tenacity of the 96-mer fiber according to the present invention was 508±108 MPa which is the highest ever reported for recombinant spider silk proteins.

In addition to the above-described wet-spinning method, the fiber can also be produced by an electrical spinning method in which voltage is applied to a solution containing the high-molecular-weight recombinant silk or recombinant silk-like protein so that the solution is extruded in the direction of the applied electric field. According this method, a micro-sized or nano-sized spider silk fiber or spider silk-like fiber can be obtained.

For example, a 12% (w/v) silk protein solution can be obtained by dissolving the recombinant silk protein in hexafluoroisopropanol (HFIP; Sigma) at room temperature for 2 days. In an electrical spinning process, a voltage of 12 kV is applied to one silk solution drop at the tip of a glass pipette including platinum wire electrodes, and when the applied electric force exceeds the surface tension of the silk solution drop, a fiber jet is formed and extruded in the direction of the applied electric field. The fiber can be collected on a glass material formed on a flat plate covered with aluminum, and the electrospun fiber may be treated with methanol to induce formation of the beta plane. The fiber may be allowed to stand at room temperature for 24 hours and then dried in air for 3 days, after which the physical properties thereof can be measured.

Next, from atomic force microscopy force curve measurements, the Young's modulus of each specimen used can be calculated. The sensitivity of the photodetector for conversion of the deflection of the cantilever is measured at room temperature (21° C.), a force-distance curve is plotted using an atomic force microscope (Dimension V; Veeco Instruments Inc., Plainview, N.Y.) together with a silicon cantilever. 20 measurements are made for each specimen, and for the measurement of Young's modulus, a modified Hertz model can be applied to each force curve (Hertz, 1882, Sneddon, 1965). The Young's modulus of each specimen is calculated directly from the obtained parameters. In the Hertz model, Young's model (E) is given by the following equations:

E=pF(1−v²)/(2a^{2tana}) (1)

F=kd (2)

wherein F, v, a, k and d indicate the force at the tip, the Poisson's ratio, the strain of the specimen, the spring constant of the cantilever, and the deflection of the cantilever, respectively. From data, including the shape and spring constant of the tip, the kind and deflection of the cantilever, and the Poisson's ratio of the specimen, the Young's modulus of the fiber can be determined.

Meanwhile, the recombinant silk protein/recombinant silk-like protein as defined herein and a fiber, filament, film, foam, sphere, nanofibril, hydrogen and the like produced therefrom may be used in the field of biotechnology and/or medicine, preferably for the manufacture of wound closure or coverage systems, suture materials for use in neurosurgery or ophthalmic surgery. Furthermore, the protein/thread may preferably be used for the manufacture of replacement materials, preferably artificial cartilage or tendon materials.

Additionally, the spider silk fiber or spider silk-like fiber of the present invention can be used in the manufacture of medical devices such as medical adhesive strips, skin grafts, replacement ligaments, and surgical mesh; and in a wide range of industrial and commercial products, such as clothing fabric, bullet-proof vest lining, container fabric, bag or purse straps, cable, rope, adhesive binding material, non-adhesive binding material, strapping material, automotive covers and parts, aircraft construction material, weatherproofing material, flexible partition material, sports equipment; and, in fact, in nearly any use of fibrils or fabric for which high tensile strength and elasticity are desired characteristics. Adaptability and use of the stable fibril product in other forms, such as a dry spray coating, bead-like particles, or use in a mixture with other compositions is also contemplated by the present invention.

The recombinant silk protein or recombinant silk-like protein of the present invention may be added to cellulose and keratin and collagen products and thus, the present invention is also directed to a paper or a skin care and hair care product, comprising cellulose and/or keratin and/or collagen and the recombinant protein of the present invention. Papers and skin care and hair care products, in which the proteins of the present invention are incorporated, show improved characteristics, in particular improved tensile strength or tear strength. Furthermore, the high-molecular-weight recombinant protein of the present invention may be used as a coating for textile and leather products, thereby conferring stability and durability to the coated product. The silk protein in particular show applicability for coating leather products, since in this case, tanning and its negative effects for environment can be avoided or at least reduced.

### EXAMPLES

Hereinafter, the present invention will be described in further detail with reference to examples. It will be obvious to a person having ordinary skill in the art that these examples are illustrative purposes only and are not to be construed to limit the scope of the present invention.

### Example 1

### Construction of vectors pSH32, pSH48, pSH64, pSH80 and pSH96 for Expression of High-Molecular-Weight Silk Proteins and Vector for Expression of Nucleotide Sequence Encoding Glycine tRNA and Preparation of Silk Proteins Having Various Molecular Weights

1-1: Construction of pSH32, pSH48, pSH64, pSH80 and pSH96

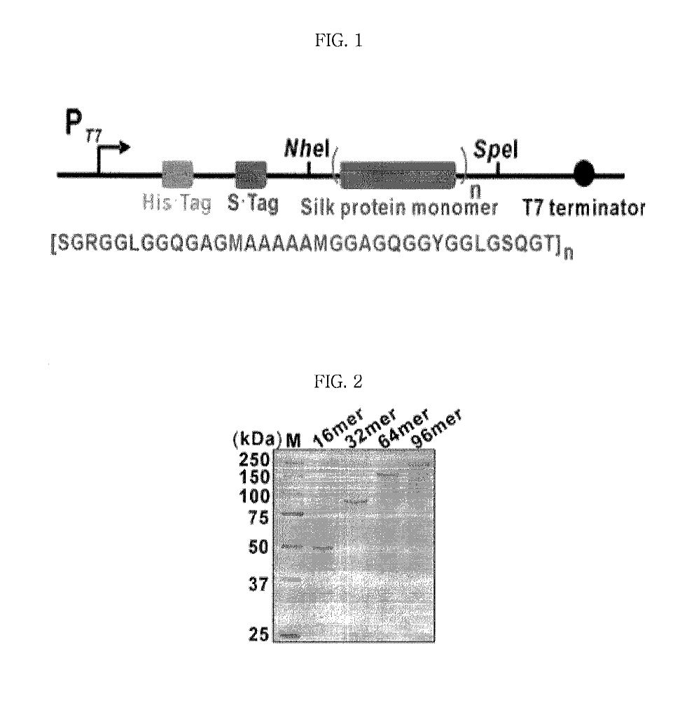

All procedures for genetic manipulation were carried out according to standard methods (Sambrook et al., Molecular cloning: a laboratory manual, 2nd Ed., Cold Spring Harbor Laboratory Press, Cold Spring Harbor, N.Y., 1989). In order to construct the recombinant plasmid pSH32, the plasmid pSH16a (Lee et al., Theories and Applications of Chem. Eng., 8(2):3969, 2002) (SEQ ID NO: 13) was digested with the restriction enzymes SpeI and NheI (New England Biolabs, USA) to obtain a 1.7-kb fragment which was then treated with the restriction enzyme SpeI and ligated to the dephosphorylated plasmid pSH16a, thereby obtaining the recombinant plasmid pSH32 comprising a nucleic acid sequence encoding a 32-mer silk protein of SEQ ID NO: 14. The orientation of the insert was checked by digestion with the restriction enzymes SpeI and NheI. In the same manner, the plasmid pSH16a was digested with the restriction enzymes SpeI and NheI to obtain a 1.7-kb fragment which was then ligated to the plasmid pSH32 digested with the restriction enzyme SpeI, thereby obtaining the recombinant plasmid pSH48. Also, the plasmid pSH32 was digested with the restriction enzymes SpeI and NheI to obtain a 3.4-kb fragment which was then ligated to the plasmid pSH32 digested with the restriction enzyme SpeI, thereby obtaining the recombinant plasmid pSH64 comprising a nucleic acid sequence encoding a 64-mer silk protein of SEQ ID NO: 15. The orientation of each insert was checked by digestion with the restriction enzymes SpeI and NheI. In addition, in the same manner, the DNA fragment of each of the plasmids pSH16a and pSH32 was inserted into the plasmid pSH64 digested with the restriction enzyme SpeI, thereby constructing the recombinant plasmid pSH80 comprising a nucleic acid sequence encoding a 80-mer silk protein of SEQ ID NO: 16, and the plasmid pSH96 comprising a nucleic acid sequence encoding a 96-mer silk protein of SEQ ID NO: 17. The orientation of the insert in each of the plasmids was checked by digestion with the restriction enzymes SpeI and NheI. FIG. 1 shows a structure for expression of the recombinant silk protein and the amino acid sequence of the repeating unit of the protein. In this regard, a nucleic acid sequence corresponding to the amino acid repeating unit of SEQ ID NO: 1 is set forth in SEQ ID NO: 18.

1-2: Construction of pTet-glyVXY Vector

All procedures for genetic manipulation were carried out according to standard methods (Sambrook et al., Molecular cloning: a laboratory manual, 2nd Ed., Cold Spring Harbor Laboratory Press, Cold Spring Harbor, N.Y., 1989). To obtain a glyVWX gene encoding glycine tRNA, PCR was performed using, as a template, a chromosome isolated from E. coli W3110 (derived from E. coli K-12, λ⁻, F⁻, prototrophic strain), and primers of SEQ ID NO: 19 and SEQ ID NO: 20.

SEQ ID NO: 19:
5′-GCTCGATATCTAACGACGCAGAAATGCGAAA-3′
SEQ ID NO: 20:
5′-CATTGGATCCTAAGATTACAGCCTGAGGCTGTG-3′

The PCR reaction was performed using Pfu polymerase (SolGent, Korea) under the following conditions: initial denaturation at 95° C. for 4 min; then 10 cycles of denaturation at 95° C. for 20 sec, annealing at 51° C. for 30 sec, and extension at 72° C., for 60 sec; and then 19 cycles of denaturation at 95° C. for 20 sec, annealing at 60° C. for 30 sec, and extension at 72° C. for 60 sec; followed by final extension at 72° C. for 5 min.

The PCR product DNA was electrophoresed on agarose gel to obtain a purified 479-bp PCR product. The PCR product was digested with the restriction enzymes BamHI and EcoRV (New England Biolabs, USA), and in order to use the promoter of a tetracycline resistant gene (tet) which can be continually used, the plasmid was also digested with the same restriction enzymes. The digested PCR product and plasmid T4 DNA were ligated with each other by ligase (Roche, Germany), and the ligated product was transformed into E. coli Top10 (F⁻ mcrA Δ(mrr-hsdRMS-mcrBC) 0lacZΔM15 ΔlacX74 recA1 araD139 Δ(ara-leu) 7697 galU galK rpsL (Str^{R}) endA1 nupG). The transformed strain was selected on LB agar solid medium (10 g/L tryptone, 5 g/L yeast extract, 5 g/L NaCl, and 15 g/L agar) containing 34 mg/L chloramphenicol, thereby constructing the recombinant plasmid pTet-glyVXY. The constructed recombinant plasmid was confirmed by digestion with restriction enzymes and base sequence analysis.

1-3: Construction of Recombinant Plasmid pTet-gly2

All procedures for genetic manipulation were carried out according to standard methods (Sambrook et al., Molecular cloning: a laboratory manual, 2nd Ed., Cold Spring Harbor Laboratory Press, Cold Spring Harbor, N.Y., 1989). To further express the glyVXY gene encoding glycine tRNA, PCR was performed using pTet-glyVXY as a template and primers of SEQ ID NOS: 21 and 22.

SEQ ID NO: 21:
5′-GGCTCGCATGCTCATGTTTGACAGCTTATCATCGA-3′
SEQ ID NO: 22:
5′-ATTGTCGACTGCTGCAGTAAGATTACAGCCTGAGGCTGTG-3′

The PCR reaction was performed using Pfu polymerase (SolGent, Korea) under the following conditions: initial denaturation at 95° C. for 3 min; then 10 cycles of denaturation at 95° C. for 20 sec, annealing at 52° C. for 30 sec, and extension at 72° C. for 50 sec; and then 19 cycles of denaturation at 95° C. for 20 sec, annealing at 62° C. for 30 sec, and extension at 72° C. for 50 sec; followed by final extension at 72° C. for 5 min.

The DNA obtained by the PCR reaction was electrophoresed on agarose gel to obtain a purified 674-bp PCRproduct. The 647-bp PCR product and the plasmid pTet-glyVXY were digested with the restriction enzymes SphI and SalI (New England Biolabs, USA) and ligated with each other by T4 DNA ligase (Roche, Germany), and the ligated product was transformed into E. coli Top10. The transformed strain was selected on LB agar solid medium (10 g/L tryptone, 5 g/L yeast extract, 5 g/L NaCl, and 15 g/L agar) containing 34 mg/L chloramphenicol, thereby constructing the recombinant plasmid pTet-gly2. The constructed recombinant plasmid was confirmed by digestion with restriction enzymes and base sequence analysis.

1-4: Preparation of Recombinant Silk Proteins Having Various Molecular Weights

In order to prepare silk proteins having various molecular weights, each of the pSH16a vector and pSH32 vector constructed in Example 1-1 was introduced into the recombinant plasmid pTet-glyVXY, obtained in Example 1-2, and E. coli BL21 (DE3) (F− ompT hsdSB(rB− mB−) gal dcm (DE3); a prophage carrying the T7 RNA polymerase gene) (New England Biolabs, USA). Meanwhile, each of the pSH64 vector and the pSH96 vector was introduced into the pTetgly2 vector, obtained in Example 1-3, and E. coli BL21 (DE3). The strains thus transformed were inoculated into LB liquid medium (10 g/L tryptone, 5 g/L yeast extract, and 5 g/L NaCl) containing 34 mg/L chloramphenicol and 25 mg/L kanamycin and were cultured with continuous shaking at 30° C. at 180 rpm. When the optical density (O.D.) measured with a spectrophotometer at a wavelength of 600 nm after inoculation of 1% of each strain reached 0.2, 0.4 or 0.6, 1 mM IPTG was added to each strain to induce the expression of silk proteins. 5 hours after induction of the expression of the silk proteins, the cultures were harvested.

For analysis of the prepared recombinant proteins, each of the harvested cultures was centrifuged at 4° C. at 10,000 g for 10 minutes to obtain cell pellets which were then dissolved in TE buffer and 5× Laemmli sample buffer. The same amount (0.024 mg) of samples were taken from the cultures using 10% SDS-PAGE and stained with Coomassie brilliant blue R250 (Bio-Rad, USA), followed by quantification with GS-710 Calibrated Imaging Densitometer (Bio-Rad, USA). The protein contents of the samples were measured by the Bradford assay using bovine serum albumin as a standard (Bradford, M. M., Anal. Biochem., 72:248, 1976).

As a result, as shown in FIG. 2, the recombinant silk proteins of 16-mer (prepared using the pSH16a vector), 32-mer (prepared using the pSH32 vector), 64-mer (prepared using the pSH64 vector) and 96-mer (prepared using the pSH96) had molecular weights of about 50.4 kDa, 100.7 kDa, 192.8 kDa and 284.9 kDa, respectively. In the prior art, it has been known that the largest of the dragline silk proteins that have been synthesized in E. coli has a molecular weight of 163 kDa and that it is difficult to produce a silk protein having a molecular weight larger than 163 kDa (Fahnestock, S. R. & Irwin, S. L., Appl. Microbiol. Biotechnol., 47:23, 1997; Vendrely, C. & Scheibel, T., Macromol. Biosci., 7:401, 2007; Lazaris, A., et al., Science, 295:472, 2002). However, according to the present invention, it was found that recombinant silk proteins having high molecular weights, such as 192.8 kDa and 284.9 kDa, can be provided by co-expressing the glycine tRNA-encoding nucleotide sequence with the expression vector as described above.

### Example 2

### Production of Spider Silk Fiber by Wet-Spinning Method—Effect of Molecular Weight on Mechanical Properties of Wet-Spun Fiber

Each of the recombinant silk proteins prepared in Example 1-2 was dissolved in hexafluoroisopropanol (HFIP; Sigma), a spinning solvent, thus preparing spinning dope solutions. Each of the dope solutions was extruded using a pump (KDS100; KD Scientific) at a rate of 1-2 ml/hr. With the silk protein concentration of the dope solutions, all the silk proteins were spun at a spider silk protein concentration of 20% (w/v), which was the maximum operational concentration for the native-sized 96-mer protein due to the solubility and viscosity. At this time, each dope solution was extruded from a 1-ml Kovax syringe through a 26-G syringe needle (Korea Vaccine Co., Ltd.) into a solidification bath containing 90% (v/v) methanol.

After the spinning process, each of the spun fibers was allowed to stand in the solidification bath for 20 minutes and was hand-drawn up to 5 times the original length. FIG. 3 shows stress-strain curves of the fibers.

Next, the fibers were dried at room temperature and continuously maintained under tension in order to prevent shrinkage and maintain the stretched lengths of the fibers during measurement.

Before the test, specimens (n=10) of the fibers were conditioned at room temperature at a relative humidity of 50% for 24 hours. The tensile test was performed with a universal tensile tester (RB302 mL, R&B Inc.) using a 100 g load cell. The gauge length was 20 mm, and the cross-head speed was 10 mm/min. Mechanical properties data are shown as means±standard deviation (n=10). Statistical analysis was performed by unpaired t-test, and P<0.05 was considered statistically significant.

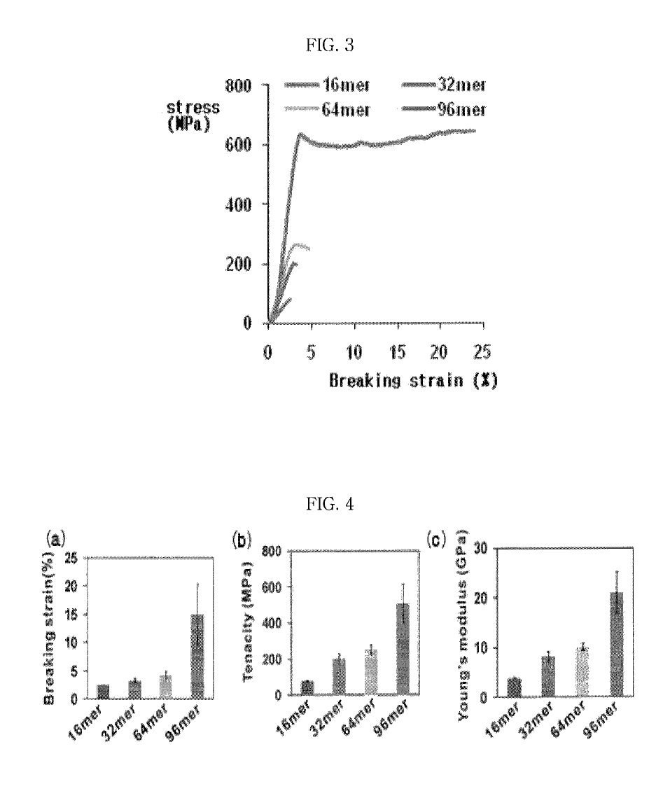

The measurement results are shown in FIG. 4a-4c. As can be seen therein, as the molecular weights of the silk proteins increased, the mechanical properties (such as breaking strain, tenacity and Young's modulus) of the fibers were improved (32-mer: breaking strain of 3.27±0.32%, tenacity of 202±25 MPa, and Young's modulus of 8.28±0.85 GPa; 64-mer: breaking strain of 4.31±0.64%, tenacity of 252±26 MPa, and Young's modulus of 10.14±0.67 GPa; and 96-mer: breaking strain of 15±5%, tenacity of 508±108 MPa, and Young's modulus of 21±4 GPa). Particularly, the spider silk fiber produced from the recombinant silk protein of 96-mer having a molecular weight reaching 284.9 kDa showed unexpected significant improvements in all breaking strain, tenacity and Young's modulus compared to the spider silk fibers produced from the recombinant silk proteins of 64-mer or less.

Specifically, the fiber spun from the recombinant silk protein of 96-mer showed a tenacity of 508±108 MPa and a breaking strain of and 15±5%, which are comparable to those of native N. clavipes dragline silk (740-1,200 MPa; 18-27%). Particularly, the Young's modulus of the 96-mer fiber was 21±4 GPa corresponding to twice that of the native dragline silk (11-14 GPa). The tenacity of the 96-mer fiber (508-108 MPa) in the present invention is the highest ever reported for recombinant spider silk proteins.

### Example 3

### Effect of Recombinant Silk Protein Concentrations of Dope Solutions on Properties of Wet-Spun Fibers

In order to examine the effects of the recombinant silk protein concentrations of dope solutions on the properties of spun fibers, each of the 16-mer, 32-mer and 64-mer proteins was spun at the maximum operational concentrations, and test results for the spun fibers were compared with the results of Example 2.

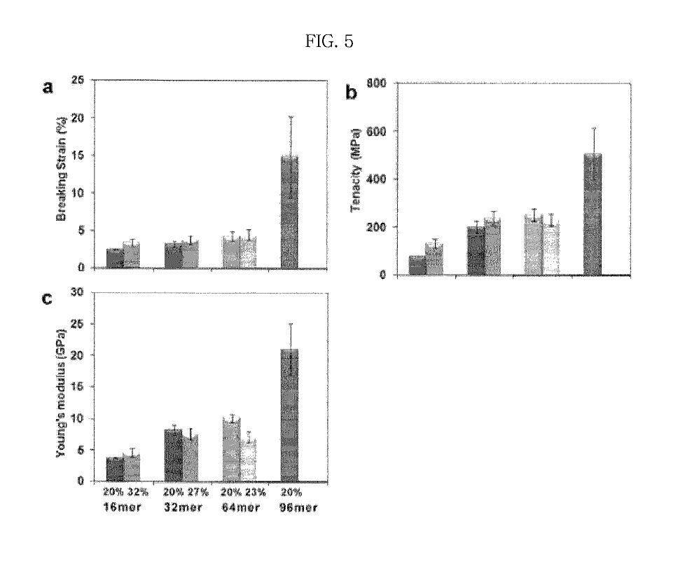

As a result, as shown in FIG. 5a-5c, when the concentration of the 16-mer protein was increased from 20% to the maximum concentration of 30%, the properties of the fiber spun from the protein were significantly improved. However, when the concentration of the 32-mer protein was increased from 20% to the maximum concentration of 27%, the breaking strain and tenacity of the fiber were increased, but these increases were not statistically significant. In addition, the maximum operational concentration of the 64-mer protein was 23%, the mechanical properties of the fiber spun from the dope solution having the 64-mer protein concentration of 23% did not significantly differ from those of the fiber spun from the dope solution having the 64-mer protein concentration of 20%.

### INDUSTRIAL APPLICABILITY

The present invention relates to a high-molecular-weight recombinant silk or silk-like protein having a molecular weight which is substantially similar to that of native silk protein, and to a micro- or nano-sized spider silk or silk-like fiber having improved physical properties, produced therefrom. The recombinant silk or silk-like protein according to the invention has high molecular weight, like dragline silk proteins from spiders, while a fiber produced therefrom has excellent physical properties compared to a fiber produced from native silk protein. Thus, the recombinant silk or silk-like protein and the spider silk or silk-like fiber produced therefrom will be highly useful in various industrial applications, including bioengineering applications and medical applications.

Although the present invention has been described in detail with reference to the specific features, it will be apparent to those skilled in the art that this description is only for a preferred embodiment and does not limit the scope of the present invention. Thus, the substantial scope of the present invention will be defined by the appended claims and equivalents thereof.

1. A high-molecular-weight recombinant silk or silk-like protein having a structure in which a peptide having a glycine content of 10% or more is repeated 64-160 times.

2. The high-molecular-weight recombinant silk or silk-like protein according to claim 1, having a structure in which the peptide is repeated 80-160 times.

3. The high-molecular-weight recombinant silk or silk-like protein according to claim 1, having a structure in which the peptide is repeated 96-160 times.

4. The high-molecular-weight recombinant silk or silk-like protein according to claim 1, wherein the peptide is a repeating peptide constituting a protein selected from the group consisting of dragline silk, elastin, silk fibroin, byssus, flagelliform silk and collagen.

5. The high-molecular-weight recombinant silk or silk-like protein according to claim 1, wherein the peptide has one of the amino acid sequences of SEQ ID NO: 1 to 11.

6. The high-molecular-weight recombinant silk or silk-like protein according to claim 5, repeating an amino acid sequence having a homology of at least 90% with the peptide.

7. A high-molecular-weight recombinant silk protein having a structure in which a peptide of SEQ ID NO: 1 is repeated 64-160 times, and having a molecular weight of 192.8-482 kDa.

8. A method for preparing a high-molecular-weight recombinant silk or silk-like protein, comprising co-expressing a gene encoding recombinant silk or silk-like protein according to claim 1 with a nucleotide sequence encoding glycine tRNA in bacteria.

9. The method according to claim 8, wherein the bacteria is E. coli.

10. A method for preparing a micro-sized or nano-sized spider silk or spider silk-like fiber, comprising spinning a dope solution containing the high-molecular-weight recombinant silk or silk-like protein according to claim 1.

11. The method according to claim 10, comprising wet-spinning a dope solution containing 20-80% (w/v) of a recombinant silk or silk-like protein.

12. The method according to claim 11, wherein the dope solution is spun in a liquid bath.

13. The method according to claim 12, wherein the liquid bath contains a liquid selected from the group consisting of methanol, ethanol, isopropanol, acetonitrile, water and aqueous ammonium sulfate.

14. A micro-sized or nano-sized spider silk or spider silk-like fiber prepared by the method according to claim 10.

15. A method for preparing a micro-sized or nano-sized spider silk fiber, comprising spinning a dope solution containing the high-molecular-weight recombinant silk protein according to claim 7.

16. A micro-sized or nano-sized spider silk fiber prepared by the method according to claim 15.

17. The micro-sized or nano-sized spider silk fiber according to claim 16, wherein the spider silk fiber has at least 252 MPa tenacity, at least 10.14 GPa Young's modulus, and at least 4.31% breaking strain.

18. A method for preparing a high-molecular-weight recombinant silk or silk-like protein, comprising co-expressing a gene encoding recombinant silk or silk-like protein according to claim 2 with a nucleotide sequence encoding glycine tRNA in bacteria.

19. A method for preparing a micro-sized or nano-sized spider silk or spider silk-like fiber, comprising spinning a dope solution containing the high-molecular-weight recombinant silk or silk-like protein according to claim 2.


SEQUENCE LISTING (22 sequences)

SEQ ID NO: 1 - protein, 35 aa, Artificial
  Repeat unit of Dragline silk protein
SGRGGLGGQGAGMAAAAAMGGAGQGGYGGLGSQGT

SEQ ID NO: 2 - protein, 5 aa, Artificial
  Repeat unit of Dragline silk protein
GPGQQ

SEQ ID NO: 3 - protein, 5 aa, Artificial
  Repeat unit of Dragline silk protein
GPGGY

SEQ ID NO: 4 - protein, 7 aa, Artificial
  Repeat unit of Dragline silk protein
GGYGPGS

SEQ ID NO: 5 - protein, 5 aa, Artificial
  Repeat unit of elastin
GVGVP

SEQ ID NO: 6 - protein, 4 aa, Artificial
  Repeat unit of elastin
VPGG

SEQ ID NO: 7 - protein, 6 aa, Artificial
  Repeat unit of elastin
APGVGV

SEQ ID NO: 8 - protein, 6 aa, Artificial
  Repeat unit of silk fibroin
GAGAGS

SEQ ID NO: 9 - protein, 5 aa, Artificial
  Repeat unit of byssus
GPGGG

SEQ ID NO: 10 - protein, 5 aa, Artificial
  Repeat unit of flagelliform silk
GPGGX

SEQ ID NO: 11 - protein, 15 aa, Artificial
  Repeat unit of collagen
GAPGAPGSQGAPGLQ

SEQ ID NO: 12 - protein, 15 aa, Artificial
  Repeat unit of collagen
GAPGTPGPQGLPGSP

SEQ ID NO: 13 - DNA, 7117 bp, Artificial
  pSH16a
tggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatcgatctcgatcccgcgaaattaatacgactcactataggggaattgtgagcggataacaattcccctctagaaataattttgtttaactttaagaaggagatatacatatgcaccatcatcatcatcattcttctggtctggtgccacgcggttctggtatgaaagaaaccgctgctgctaaattcgaacgccagcacatggacagcccagatctgggtaccgacgacgacgacaaggccatggctgatatcggatccatggctagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagtggatccgaattcgagctccgtcgacaagcttgcggccgcactcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggat

SEQ ID NO: 14 - DNA, 3600 bp, Artificial
  32mer silk protein nucleic acid sequence
atgcaccatcatcatcatcattcttctggtctggtgccacgcggttctggtatgaaagaaaccgctgctgctaaattcgaacgccagcacatggacagcccagatctgggtaccgacgacgacgacaaggccatggctgatatcggatccatggctagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagtggatccgaattcgagctccgtcgacaagcttgcggccgcactcgagcaccaccaccaccaccactgagatccggctgctaa

SEQ ID NO: 15 - DNA, 6960 bp, Artificial
  64mer silk protein nucleic acid sequence
atgcaccatcatcatcatcattcttctggtctggtgccacgcggttctggtatgaaagaaaccgctgctgctaaattcgaacgccagcacatggacagcccagatctgggtaccgacgacgacgacaaggccatggctgatatcggatccatggctagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagtggatccgaattcgagctccgtcgacaagcttgcggccgcactcgagcaccaccaccaccaccactgagatccggctgctaa

SEQ ID NO: 16 - DNA, 8640 bp, Artificial
  80mer silk protein nucleic acid sequence
atgcaccatcatcatcatcattcttctggtctggtgccacgcggttctggtatgaaagaaaccgctgctgctaaattcgaacgccagcacatggacagcccagatctgggtaccgacgacgacgacaaggccatggctgatatcggatccatggctagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagtggatccgaattcgagctccgtcgacaagcttgcggccgcactcgagcaccaccaccaccaccactgagatccggctgctaa

SEQ ID NO: 17 - DNA, 10320 bp, Artificial
  96mer silk protein nucleic acid sequence
atgcaccatcatcatcatcattcttctggtctggtgccacgcggttctggtatgaaagaaaccgctgctgctaaattcgaacgccagcacatggacagcccagatctgggtaccgacgacgacgacaaggccatggctgatatcggatccatggctagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagcggtcgcggcggtctgggtggccagggtgcaggtatggcggctgcggctgcaatgggcggtgctggccaaggtggctacggcggcctgggttctcagggtactagtggatccgaattcgagctccgtcgacaagcttgcggccgcactcgagcaccaccaccaccaccactgagatccggctgctaa

SEQ ID NO: 18 - DNA, 105 bp, Artificial
  nucleic acid sequence encoding amino acid sequence of SEQ Number 1
agcggtcgcggcggtctgggtggccagggtgcaggtatggcggctgcggctgcaatgggcggtgctggccaaggtggctacggcggcctgggttctcagggtact

SEQ ID NO: 19 - DNA, 31 bp, Artificial
  PCR primer
gctcgatatctaacgacgcagaaatgcgaaa

SEQ ID NO: 20 - DNA, 33 bp, Artificial
  PCR primer
cattggatcctaagattacagcctgaggctgtg

SEQ ID NO: 21 - DNA, 35 bp, Artificial
  PCR primer
ggctcgcatgctcatgtttgacagcttatcatcga

SEQ ID NO: 22 - DNA, 40 bp, Artificial
  PCR primer
attgtcgactgctgcagtaagattacagcctgaggctgtg